(19) United States
(12) Patent Application Publication
(10) Pub. No.: US 2019/0209473 A1
(43) Pub. Date: Jul. 11, 2019

(54) FOAMING CREAM EMULSIONS

(71) Applicant: Founders Science Group, Taunton, MA (US)

(72) Inventors: Robert P. Munroe, North Dighton, MA (US); Ronald M. Gurge, Franklin, MA (US)

(21) Appl. No.: 16/240,249

(22) Filed: Jan. 4, 2019

Related U.S. Application Data
(60) Provisional application No. 62/614,107, filed on Jan. 5, 2018.

(51) Int. Cl. A61K 9/12; A61K 9/107; A61K 47/10; A61K 47/14; A61K 47/26; A61K 9/06; A61K 45/06; A61K 8/04; A61K 8/06; A61K 8/92

(52) U.S. Cl. CPC A61K 9/124; A61K 9/107; A61K 47/10; A61K 47/14; A61K 47/26; A61P 17/16; A61K 45/06; A61K 8/046; A61K 8/062; A61K 8/922; A61K 2800/87; A61K 9/06

(57) ABSTRACT

Disclosed are self-foaming compositions, comprising a heavy cream product and a propellant, which are admixed, and may be used to produce a foam for use in pharmaceutical, dermatological, and cosmetic applications.

### RELATED APPLICATION

This application claims the benefit of priority to U.S. Provisional Patent Application No. 62/614,107, filed on Jan. 5, 2018.

### BACKGROUND OF THE INVENTION

Cosmetic foams are usually dispersed systems of liquids and gases (or propellants), where the liquid represents the dispersant and the gas represents the dispersed substance. Foams of low-viscosity liquids are temporarily stabilized by surface-active substances (surfactants, foam stabilizers). Due to their large internal surface area, such surfactant foams have a high adsorption capacity, which is utilized, for example, in cleaning and washing operations. Accordingly, cosmetic foams are used, in particular, in the fields of cleansing, for example as shaving foam, and of hair care.

When these products are dispensed the gas pressure in its dispensing container expels these the liquid and gas such that the gas rapidly escapes and liquid “instantly” foams. However, when too much gas is dispensed by the dispensing container (such as in inverted orientation or due to improper mixing), the foam cannot be properly dispensed from the container. Further, such dispensing systems are only effective for dispensing products having low viscosity. Thus, there remains a need in the art for new compositions for producing foam products, especially those that are highly viscous, and methods of producing such foams.

### SUMMARY

Compositions comprising a heavy cream product and a liquefied or compressed gas propellant are disclosed herein. In some embodiments, the composition comprises 88-97 wt % of the heavy cream product, and, as such, also comprises 3-12 wt % of the liquefied or compressed gas propellant. In certain embodiments, the heavy cream product and the liquefied or compressed gas propellant are admixed. In certain embodiments, the composition is self-foaming.

Also provided herein are packaged composition, comprising a dispenser; and the self-foaming compositions disclosed herein; wherein the composition is contained within the dispenser; and the composition forms a non-flammable foam upon dispensing.

Also provided herein are methods dispensing a self-foaming composition from a packaged composition of the invention such that a composition is released from the dispenser, and forms a non-flammable foam upon dispensing.

### BRIEF DESCRIPTION OF THE DRAWINGS

FIG. 1 shows a standard aerosol configuration.

FIG. 2 shows an exemplary bag-on-valve aerosol configuration.

### DETAILED DESCRIPTION

Provided herein are compositions comprising a heavy cream product and propellant that are admixed. In some embodiments, the composition may be used to produce a foam for use in pharmaceutical, dermatological, and cosmetic applications. In certain embodiments, the composition is self-foaming. Where the composition is self-foaming, the composition may form a non-flammable foam concurrently or substantially concurrently with dispensing from its packaging and/or container.

The heavy cream product used in the composition of the invention comprises an emulsion, preferably an oil-in-water emulsion. An oil-in-water emulsion is an emulsion in which oil is the dispersed liquid and an aqueous solution is the continuous phase. Suitable oil-in-water emulsions comprise a high emollient, high viscosity cream, lotion, or ointment (e.g., a natural oil, a synthetic oil, or a combination thereof) and water. In certain embodiments, the high emollient, high viscosity cream, lotion, or ointment comprises one or more fatty alcohols (e.g., cetostearyl alcohol, cetyl stearyl alcohol, cetyl alcohol, myristyl alcohol), polyalkylene glycol monoalkyl ether, phosphates thereof, alkylene oxide polyol adducts, ethylhexyl palmitate, coco-caprylate/caprate, C₁₂-C₁₅-alkyl ethylhexanoate, isononyl isononanoate, or a combination thereof.

Typically, the oil-in-water emulsion used in the disclosed composition comprises from 10%-90% water. For example, the oil-in-water emulsion comprises from about 10% to about 90% water, from about 15% to about 90% water, from about 20% to about 90% water, from about 25% to about 90% water, from about 30% to about 90% water, from about 35% to about 90% water, from about 40% to about 90% water, from about 45% to about 90% water, from about 50% to about 90% water, from about 55% to about 90% water, from about 60% to about 90% water, from about 65% to about 90% water, from about 70% to about 90% water, from about 10% to about 70% water, from about 15% to about 70% water, or from about 20% to about 90% water. In certain embodiments, the oil-in-water emulsion comprises from 20% to about 70% water, such as about 20% water, about 25% water, about 30% water, about 35% water, about 40% water, about 45% water, about 50% water, about 55% water, about 60% water, about 65% water, or about 70% water.

Propellants are used to generate and administer the foamable composition as a foam. Propellants comprises a liquefied or compressed gaseous substance. In general, the propellant makes up about 1-15 wt % of the composition of the invention. For example, in some embodiments, the composition comprises from about 1 wt % to about 15 wt %, from about 2 wt % to about 15 wt %, from about 3 wt % to about 15 wt %, from about 4 wt % to about 15 wt %, from about 5 wt % to about 15 wt %, from about 6 wt % to about 15 wt %, from about 7 wt % to about 15 wt %, from about 8 wt % to about 15 wt %, from about 9 wt % to about 15 wt %, from about 10 wt % to about 15 wt %, from about 11 wt % to about 15 wt %, from about 12 wt % to about 15 wt %, from about 1 wt % to about 12 wt %, from about 2 wt % to about 12 wt %, or from about 3 wt % to about 12 wt % of a liquefied or compressed gas propellant. In certain embodiments, the composition comprises 3 wt % to about 12 wt % of the liquefied or compressed gas propellant, such as, for example, about 3 wt %, about 4 wt %, about 5 wt %, about 6 wt %, about 7 wt %, about 8 wt %, about 9 wt %, about 10 wt %, about 11 wt %, or about 12 wt % of the liquefied or compressed gas propellant.

Both flammable and nonflammable the liquefied or compressed gas propellant can be used in the compositions of the invention. Flammable propellants include volatile hydrocarbons (e.g., propane, n-butane, isobutane, isobutene, isopentane, and combinations thereof) and volatile ethers (e.g., dimethyl ether or methyl ethyl ether). Alternatively the liquefied or compressed gas propellant can be a green propellant, such as a hydrofluoroalkane (e.g., 1,1,1,2,3,3,3-heptafluoropropane) or a hydrofluoroalkene (e.g., 1,3,3,3-tetrafluoropropene). Green propellants are less toxic to the environment.

The compositions hereof may also contain a surfactant. Without being limited by theory, it is believed that the surfactant assists in providing rheological characteristics to the composition, which contribute to the stability of the composition.

Surfactants of the present invention may be an ionic surfactant (cationic or anionic) or a nonionic surfactant. Exemplary surfactants include is cetostearyl alcohol, polysorbate 60, PEG-150 stearate and/or steareth-20, or combinations thereof.

The compositions of the present invention may further optionally comprise a variety of pharmaceutical or cosmetic ingredients, which are added in order to fine-tune the consistency of the formulation, protect the formulation components from degradation and oxidation, and bestow their cosmetic acceptability. Such excipients, may be selected, for example, from the group consisting of diglycerides, triglycerides, stabilizing agents, antioxidants, humectants, flavoring, colorant and odorant agents and other formulation components, used in the art of pharmaceutical and cosmetic formulary.

### Chelators

The compositions of the present invention may also contain a safe and effective amount of a chelator or chelating agent. As used herein, “chelator” or “chelating agent” means an active agent capable of removing a metal ion from a system by forming a complex so that the metal ion cannot readily participate in or catalyze chemical reactions. The inclusion of a chelating agent is especially useful for providing protection against UV radiation which can contribute to excessive scaling or skin texture changes and against other environmental agents which can cause skin damage.

A safe and effective amount of a chelating agent may be added to the compositions of the subject invention, preferably from about 0.1% to about 10%, more preferably from about 1% to about 5%, of the composition. Exemplary chelators that are useful herein are disclosed in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Oct. 31, 1995. The US patent and published international applications cited in this paragraph are incorporated by reference.

### Flavonoids

The compositions of the present invention may optionally contain a flavonoid compound. Flavonoids are disclosed in U.S. Pat. Nos. 5,686,082 and 5,686,367, both of which are incorporated by reference. Flavonoids suitable for use in the present invention are flavanones selected from unsubstituted flavanones, mono-substituted flavanones, and mixtures thereof; chalcones selected from unsubstituted chalcones, mono-substituted chalcones, di-substituted chalcones, tri-substituted chalcones, and mixtures thereof; flavones selected from unsubstituted flavones, mono-substituted flavones, di-substituted flavones, and mixtures thereof; one or more isoflavones; coumarins selected from unsubstituted coumarins, mono-substituted coumarins, di-substituted coumarins, and mixtures thereof; chromones selected from unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with hydroxyl, C₁-C₈ alkyl, C₁-C₄ alkoxyl, glycoside, and the like, or a mixture of these substituents.

Examples of suitable flavonoids include, but are not limited to, unsubstituted flavanone, mono-hydroxy flavanones (e.g., 2′-hydroxy flavanone, 6-hydroxy flavanone, 7-hydroxy flavanone, etc.), mono-alkoxy flavanones (e.g., 5-methoxy flavanone, 6-methoxy flavanone, 7-methoxy flavanone, 4′-methoxy flavanone, etc.), unsubstituted chalcone (especially unsubstituted trans-chalcone), mono-hydroxy chalcones (e.g., 2′-hydroxy chalcone, 4′-hydroxy chalcone, etc.), di-hydroxy chalcones (e.g., 2′,4-dihydroxy chalcone, 2′,4′-dihydroxy chalcone, 2,2′-dihydroxy chalcone, 2′,3-dihydroxy chalcone, 2′,5′-dihydroxy chalcone, etc.), and tri-hydroxy chalcones (e.g., 2′,3′,4′-trihydroxy chalcone, 4,2′,4′-trihydroxy chalcone, 2,2′,4′-trihydroxy chalcone, etc.), unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), unsubstituted coumarin, 4-hydroxy coumarin, 7-hydroxy coumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, and mixtures thereof.

### Anti-Inflammatory Agents

A safe and effective amount of an anti-inflammatory agent may be added to the compositions of the present invention, for example, from about 0.1% to about 10%, more preferably from about 0.5% to about 5%, of the composition. The anti-inflammatory agent enhances the skin appearance benefits of the present invention, e.g., such agents contribute to a more uniform and acceptable skin tone or color. The exact amount of anti-inflammatory agent to be used in the compositions will depend on the particular anti-inflammatory agent utilized since such agents vary widely in potency.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

A second class of anti-inflammatory agents that is useful in the compositions includes the nonsteroidal anti-inflammatory agents. The variety of compounds encompassed by this group are well-known to those skilled in the art. For detailed disclosure of the chemical structure, synthesis, side effects, etc. of non-steroidal anti-inflammatory agents, one may refer to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974).

Specific non-steroidal anti-inflammatory agents useful in the composition include, but are not limited to:
1) the oxicams, such as piroxicam, isoxicam, tenoxicam, sudoxicam, and CP-14,304;
2) the salicylates, such as aspirin, disalcid, benorylate, trilisate, safapryn, solprin, diflunisal, and fendosal;
3) the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;
4) the fenamates, such as mefenamic, meclofenamic, flufenamic, niflumic, and tolfenamic acids;
5) the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and
6) the pyrazoles, such as phenylbutazone, oxyphenbutazone, feprazone, azapropazone, and trimethazone.

Mixtures of these non-steroidal anti-inflammatory agents may also be employed, as well as the dermatologically acceptable salts and esters of these agents. For example, etofenamate, a flufenamic acid derivative, is particularly useful for topical application. Of the nonsteroidal anti-inflammatory agents, ibuprofen, naproxen, flufenamic acid, etofenamate, aspirin, mefenamic acid, meclofenamic acid, piroxicam and felbinac are preferred; ibuprofen, naproxen, ketoprofen, etofenamate, aspirin and flufenamic acid are more preferred.

Finally, so-called “natural” anti-inflammatory agents may be useful herein. Such agents may suitably be obtained as an extract by suitable physical and/or chemical isolation from natural sources (e.g., plants, fungi, by-products of microorganisms) or can be synthetically prepared. For example, candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commiphora, particularly Commiphora Mukul), kola extract, chamomile, red clover extract, and sea whip extract, may be used.

Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred.

### Anti-Cellulite Agents

The compositions of the present invention may also contain a safe and effective amount of an anti-cellulite agent. Suitable agents may include, but are not limited to, xanthine compounds (e.g., caffeine, theophylline, theobromine, and aminophylline).

### Topical Anesthetics

The compositions of the present invention may also contain a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

### Tanning Agents

The compositions of the present invention may contain a tanning agent. For example, the compositions of the present invention may include dihydroxyacetone as an artificial tanning agent. When present, the compositions may contain from about 0.1% to about 20%, from about 2% to about 7%, or from about 3% to about 6%, by weight of the composition, of the tanning agent.

### Skin-Lightening Agents

The compositions of the present invention may contain a skin-lightening agent. When used, the compositions may contain from about 0.1% to about 10%, from about 0.2% to about 5%, or from about 0.5% to about 2%, by weight of the composition, of a skin-lightening agent. Suitable skin lightening agents include those known in the art, including kojic acid, arbutin, ascorbic acid and derivatives thereof (e.g., magnesium ascorbyl phosphate or sodium ascorbyl phosphate), and extracts (e.g., mulberry extract, placental extract). Skin lightening agents suitable for use herein also include those described in PCT publications WO 95/34280 and WO 95/23780 (both of which are incorporated by reference).

### Skin-Soothing and Skin-Healing Agents

The compositions of the present invention may comprise a skin soothing or skin-healing agent. Skin soothing or skin-healing agents suitable for use herein include panthenoic acid derivatives (including panthenol, dexpanthenol, ethyl panthenol), aloe vera, allantoin, bisabolol, and dipotassium glycyrrhizinate. A safe and effective amount of a skin soothing or skin healing agent may be added to the present composition, for example, from about 0.1% to about 30%, from about 0.5% to about 20%, or from about 0.5% to about 10%, by weight of the composition formed.

### Antimicrobial and Antifungal Agents

The compositions of the present invention may contain an antimicrobial or antifungal agent. Such agents are capable of destroying microbes, preventing the development of microbes or preventing the pathogenic action of microbes. A safe and effective amount of an antimicrobial or antifungal agent may be added to the present compositions, for example, from about 0.001% to about 10%, from about 0.01% to about 5%, or from about 0.05% to about 2%.

Examples of antimicrobial and antifungal agents include B-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione and clotrimazole.

Additionally antimicrobial peptides can be used.

### Sunscreen Agents

Exposure to ultraviolet light can result in excessive scaling and texture changes of the stratum corneum. Therefore, the compositions of the subject invention may optionally contain a sunscreen agent. As used herein, “sunscreen agent” includes both sunscreen agents and physical sunblocks. Suitable sunscreen agents may be organic or inorganic.

Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of, for example, from about 0.1% to about 20%, preferably from about 0.5% to about 10%, more preferably from about 1% to about 5%, by weight of the composition.

A wide variety of conventional organic sunscreen agents are suitable for use herein. Sagarin, et al., at Chapter VIII, pages 189 et seq., of Cosmetics Science and Technology (1972), discloses numerous suitable agents. Specific suitable sunscreen agents include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane.

### Conditioning Agents

The compositions of the present invention may contain a conditioning agent selected from humectants, moisturizers, or skin conditioners. A variety of these materials can be employed and each can be present at a level of from about 0.01% to about 20%, from about 0.1% to about 10%, or from about 0.5% to about 7% by weight of the composition. These materials include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fucose, glucosamine); hyaluronic acid; lactamide monoethanolamine; acetamide monoethanolamine; panthenol; allantoin; and mixtures thereof. Also useful herein are the propoxylated glycerols described in U.S. Pat. No. 4,976,953 (incorporated by reference), to Orr et al, issued Dec. 11, 1990.

### Thickening Agent (Including Thickeners and Gelling Agents)

The compositions of the present invention can contain one or more thickening agents, for example, from about 0.1% to about 5%, from about 0.1% to about 4%, or from about 0.25% to about 3%, by weight of the composition.

Non-limiting classes of thickening agents for use in the compositions of the invention include those selected from the following: carboxylic acid polymers (such as those described in U.S. Pat. No. 5,087,445, to Haffey et al, issued Feb. 11, 1992; U.S. Pat. No. 4,509,949, to Huang et al, issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80); cross-linked polyacrylate polymers (such as those described in U.S. Pat. No. 5,100,660, to Hawe et al, issued Mar. 31, 1992; U.S. Pat. No. 4,849,484, to Heard, issued Jul. 18, 1989; U.S. Pat. No. 4,835,206, to Farrar et al, issued May 30, 1989; U.S. Pat. No. 4,628,078 to Glover et al issued Dec. 9, 1986; U.S. Pat. No. 4,599,379 to Flesher et al issued Jul. 8, 1986; and EP 228,868, to Farrar et al, published Jul. 15, 1987); polyacrylamide polymers (such as nonionic polyacrylamide polymers including substituted branched or unbranched polymers and multi-block copolymers of acrylamides and substituted acrylamides with acrylic acids and substituted acrylic acids); polysaccharides (which refers to gelling agents that contain a backbone of repeating sugar (i.e., carbohydrate) units, including cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof); and gums (such as acacia, agar, algin, alginic acid, ammonium alginate, amylopectin, calcium alginate, calcium carrageenan, camitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof). All of the US patents disclosed in this paragraph are incorporated by reference.

### Packaged Compositions

In some embodiments, the compositions of the invention are provided as a packaged composition. In some such embodiments, the packaged composition comprises a dispenser that dispenses the composition of the invention such that the composition forms a non-flammable foam upon dispensing. In certain embodiments, the non-flammable foam is formed upon dispensing from any orientation of the dispenser. In other embodiments, the non-flammable foam is formed upon dispensing without external mixing or shaking.

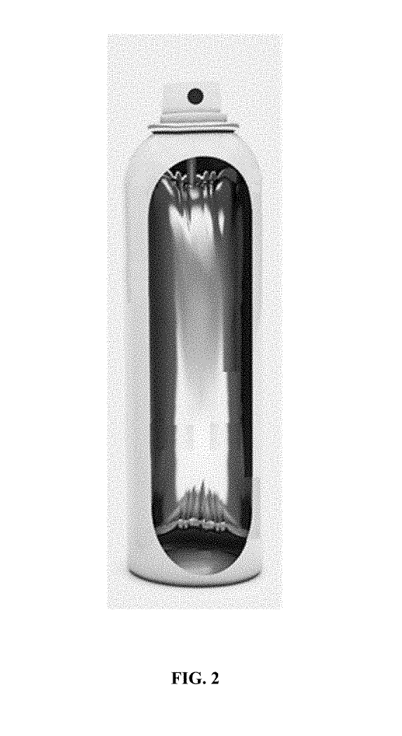

In some embodiments, the dispenser is a pressurized dispenser (e.g., an aerosol dispenser). In certain embodiments, the aerosol dispenser is a bag-on-valve aerosol dispenser. An exemplary bag-on-valve aerosol dispenser is shown in FIG. 2. In some such embodiments, a gas (e.g., air or an inert gas) surrounds the outside of the bag of the bag on valve aerosol dispenser, wherein the pressure outside of the bag is greater than or equal to the vapor pressure of the propellant.

The bag-on-valve aerosol dispensers disclosed herein comprise a bag contained in a pressurized container, such as a metal can, wherein the bag holds a composition of the invention. The pressure outside of the bag is used to dispense the compositions of the invention from within the bag. Prior to filling the bag with a composition of the invention, the heavy cream product and the propellant are mixed to create a homogenous mixture. Upon dispensure, the gas outside of the bag forces the heavy cream to release from the bag and out of the container at which time, the propellant is also released and instantaneously foams the composition.

In one embodiment, the bag-on-valve aerosol container comprises an aluminum can. Aluminum cans having a diameter from about 20 mm to about 60 mm, and a height from about 40 mm to about 150 mm, are preferred, but other sizes will be suitable depending on the intended use.

Inside of and crimped to the aluminum can is a “bag on valve” closure system. The size of the bag on valve depends on the desired fill size and the can size.

The void around the bag and inside of the can may be charged with an amount of dispensing gas, e.g., air or inert gas, as detailed above. The pressure of the gas can be from about 2 psi to about 30 psi.

After assemblage, crimping, and gassing the aluminum can, the composition disclosed herein is charged through the valve stem. The can/valve assembly is fitted with an actuator or button to depress the valve and allow product to be dispensed.

Once assembled the pressure outside the bag is used to dispense the material from the bag, while the propellant mixed in the product (prior to filling) is used to foam the product that is dispensed.

Since the pressure outside of the bag is maintained, the heavy cream product does not foam inside of the bag. Foaming only takes place when the heavy cream product is released under normal, atmospheric conditions.

One advantage of the bag-on-valve aerosol containers disclosed herein is that foaming occurs concurrently or nearly concurrently upon release of the compositions of the invention; such aerosol containers do not require any additional energy (e.g., rubbing or warming) to foam. Without being bound by any one particular theory, these advantages may be attributed in part to the ratio of the propellant to heavy cream product and types of propellants used in the compositions of the invention. Another advantage of the bag-on-valve aerosol containers disclosed herein is that they properly dispense the foam when the container is disposed in any orientation.

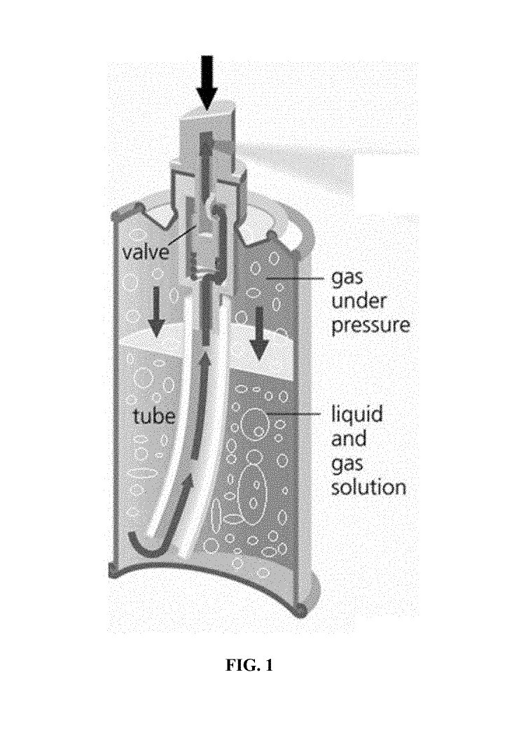

In contrast to the bag-on-valve aerosol containers disclosed herein, a standard aerosol container (FIG. 1) would allow the propellant and the heavy cream product to mix in order to form the foam. In particular, standard aerosol containers form the foam inside the can prior to dispensure. That is, the propellant is the same as the foaming gas. Accordingly, propellant can be dispensed without the product, which is especially true for products that are highly viscous or if the orientation of the container is not properly maintained.

### Pharmaceutical Applications

By including an appropriate therapeutic agent in the foamable carrier, the foam composition of the present invention is useful in the therapy of a variety of dermatological disorders (also termed “dermatoses”), including, in a non-limiting exemplary manner: Contact Dermatitis (e.g., Atopic Dermatitis, Seborrheic Dermatitis, Nummular Dermatitis, Chronic Dermatitis Of The Hands And Feet, Generalized Exfoliative Dermatitis, Stasis Dermatitis, Lichen Simplex Chronicus); Bacterial Infections (e.g., Cellulitis, Acute Lymphangitis, Lymphadenitis, Erysipelas, Cutaneous Abscesses, Necrotizing Subcutaneous Infections, Staphylococcal Scalded Skin Syndrome, Folliculitis, Furuncles, Hidradenitis Suppurativa, Carbuncles, Paronychial Infections, Erythrasma); Fungal Infections (e.g., Dermatophyte Infections, Yeast Infections); Parasitic Infections (e.g., Scabies, Pediculosis, Creeping Eruption); Viral Infections; Disorders of Hair Follicles and Sebaceous Glands (e.g., Acne, Rosacea, Perioral Dermatitis, Hypertrichosis (Hirsutism), Alopecia, including male pattern baldness, alopecia areata, alopecia universalis and alopecia totalis, Pseudofolliculitis Barbae, Keratinous Cyst); Scaling Papular Diseases (e.g., Psoriasis, Pityriasis Rosea, Lichen Planus, Pityriasis Rubra Pilaris); Benign Tumors (e.g., Moles, Dysplastic Nevi, Skin Tags, Lipomas, Angiomas, Pyogenic Granuloma, Seborrheic Keratoses, Dermatofibroma, Keratoacanthoma, Keloid); Malignant Tumors (e.g., Basal Cell Carcinoma, Squamous Cell Carcinoma, Malignant Melanoma, Paget's Disease Of The Nipples, Kaposi's Sarcoma); Reactions To Sunlight (e.g., Sunburn, Chronic Effects of Sunlight; Photosensitivity); Bullous Diseases (e.g., Pemphigus, Bullous Pemphigoid, Dermatitis Herpetiformis, Linear Immunoglobulin A Disease); Pigmentation Disorders (e.g., Hypopigmentation, Vitiligo, Albinism, Postinflammatory hypopigmentation, Hyperpigmentation, Melasma (chloasma), Drug-induced hyperpigmentation, Postinflammatory hyperpigmentation); Disorders of Cornification (e.g., Ichthyosis, Keratosis Pilaris, Calluses And Corns, Actinic keratosis); Pressure Sores; Disorders of Sweating; and Inflammatory reactions (e.g., Drug Eruptions, Toxic Epidermal Necrolysis, Erythema Multiforme, Erythema Nodosum, Granuloma Annulare).

In one or more embodiments of the present invention, the foam composition of the present invention is useful in the therapy of non-dermatological disorders, which respond to transdermal delivery of an active agent. By way of example, such disorders include localized pain in general, as well as joint pain, muscle pain, back pain, rheumatic pain, arthritis, ostheoarthritis and acute soft tissue injuries and sports injuries. Other disorders of this class include conditions, which respond to hormone therapy, such as hormone replacement therapy, transdermal nicotine administration, and other respective disorders, known in the art of drug delivery. The foam composition of the present invention is also useful in the delivery of local anesthetic agents.

### Active Pharmaceutical Agents (Drugs)

The active pharmaceutical agents, also referred to as “drug(s)”, may consist of a single drug or a combination of drugs that can be dissolved in the water phase or the hydrophobic phase of the carrier composition. Examples of such drugs are antibiotic, antibacterial, antifungal, antiviral, antiinflammatory, anesthetic, analgesic, antiallergic, corticosteroid, retinoid and antiproliferative medications and mixtures thereof at any proportion. The concentration of drugs may be adopted to exert a therapeutic effect on a disease when applied to an afflicted area.

### Antibacterial Agents

One important class of drugs comprises antibacterial agents. It is well known that bacterial infections are involved in a variety of superficial disorders of the skin, eye, mucosal membrane, oral cavity, vagina and rectum. The antibacterial drug can be active against gram positive and gram-negative bacteria, protozoa, aerobic bacteria and unaerobic ones.

By way of example, the antibacterial drugs can be selected from the group of chloramphenicol, tetracyclines, synthetic and semi-synthesic penicillins, beta-lactames, quinolones, fluoroquinolnes, macrolide antibiotics, metronidazlole and its derivatives and analogs, dicarboxylic acids, such as azelaic acid, slicylates, peptide antibiotics, cyclosporines and any combination thereof at a therapeutically effective concentration. Another group of antibacterial agents which is non-specific, comprises strong oxidants and free radical liberating compounds, such as hydrogen peroxide, bleaching agents (e.g., sodium, calcium or magnesium hypochloride and the like) iodine, chlorohexidine and benzoyl peroxide.

Antibacterial compositions according to the present invention may be used to treat infections of the skin. An example of a very common skin infection is acne, which involve infestation of the sebaceous gland with P. acnes, as well staphylococus aurus and pseudomonas. Various antibacterial agents have been utilized to treat acne, however, their efficacy is limited due to their low penetration into the hydrophobic environment of the skin layers and sebaceous glands. The composition of the present invention, comprising a hydrophobic component, would facilitate an enhanced rate of penetration. Furthermore, the intrinsic antibacterial and antiinflammatory effects of the foam adjuvant agents, i.e., fatty alcohols and acids, provides a combined effect that should result in a better therapeutic response to treatment.

The composition of the present invention is particularly useful and beneficial in the prevention and treatment of secondary infections, accompanying skin-structure damage, such as in cuts, wounds, burns and ulcers. In all such cases, the present formulation is easy to use, being in foam state when applied and becoming liquid instantly upon rubbing onto the skin.

While being useful in the prevention and treatment of infections, the antibacterial foam of the present invention is also applicable for decontaminating areas, afflicted with bacterial warfare organisms, such as anthrax and smallpox.

The same advantage is expected when the composition of the present invention is topically applied to mucosal membranes, the oral cavity, the vagina and the rectum.

### Antifungal Agents

Fungal infections are another object of treatment using the composition of the present invention. Superficial fungal infection of the skin is one of the commonest skin diseases seen in general practice. Dermatophytosis is one of the most common superficial fungal infection of the skin. It is caused by a group of fungi, which are capable of metabolizing the keratin of human epidermis, nails or hair. There are three genera of dermatophytes that may cause dermatophytosis, i.e, microsporum, trichophyton and epidermophyton.

Candidiasis is an infection caused by the yeast like fungus candida albicans or occasionally other species of candida. Clinical syndromes of candidiasis include: (a) oral candidiasis (oral thrush); (b) candidiasis of the skin and genital mucous membrane; and (c) candida paronychia, which inflicts the nail.

The pharmaceutical composition may comprise an antifungal drug, which is active against dermatophytes and candida, selected from the group of, but not limited to azoles, diazoles, triazoles, miconazole, fluconazole, ketoconazole, clotrimazole, itraconazole griseofulvin, ciclopirox, amorolfine, terbinafine, Amphotericin B, potassium iodide, flucytosine (5FC) and any combination thereof at a therapeutically effective concentration.

It is useful, for example for the treatment of tinea corporis, tinea pedis, tinea rubrum, tinea unguium, tinea cruris, tinea barbae and tinea versicolor, as well as yeast Infections, such as candidiasis, and candidal vaginitis.

### Antiviral Agents

The composition of the present invention is particularly beneficial in the case of viral infections. Cold sores are caused by the herpes simplex Type 1 virus and are sometimes referred to as facial herpes. Mollusca are small viral growths that appear singly or in groups on the face, trunk, lower abdomen, pelvis, inner thighs, or penis. Shingles (herpes zoster), which usually only occurs once in a lifetime, appears as a rash (clusters of blisters with a red base). It is caused by the same virus responsible for chickenpox. Warts are a common, benign skin tumor caused by viral infection.

Viral infections are currently treated with various antiviral agents, as summarized in the following table:

| Drug | Virus(es) | Chemotype |
|---|---|---|
| Vidarabine | Herpes viruses | Nucleoside analogue |
| Acyclovir | Herpes simplex (HSV) | Nucleoside analogue |
| Gancyclovir | Cytomegalovirus (CMV) | Nucleoside analogue |
| Nucleoside analogue reverse | Retroviruses (e.g., HIV) | Nucleoside analogue |
| transcriptase inhibitors | | |
| (NRTI) (e.g, AZT | | |
| (Zidovudine), ddl | | |
| (didanosine), ddC | | |
| (Zalcitabine), d4T (stavudine) | | |
| and 3TC (Larnivudine) | | |
| Non-Nucleoside analogue | Retroviruses (e.g., HIV) | Nucleoside analogue |
| reverse transcriptase | | |
| inhibitors (NNRTI) (e.g., | | |
| Nevirapine and delavirdine) | | |
| Protease Inhibitors (e.g., | HIV | Peptide Analogues |
| Saquinavir, Ritonavir, | | |
| Indinavir, and Nelfinavir) | | |
| Ribavirin | Broad Spectrum (e.g., HCV, | Triazole carboxamide |
| | HSV, measles, mumps, and | |
| | Lassa Fever) | |
| Amantadine/Rimantadine | Influenza A strains | Tricyclic amine |
| Interferons | Hepatitis B and C | Protein |

Any of the above antiviral drugs, in a therapeutically effective concentration, can be incorporated in the foam composition of the present invention. The composition of the present invention, which comprises a hydrophobic solvent, would facilitate an enhanced rate of penetration and better topical distribution of any of the above listed antiviral drugs. Furthermore, the intrinsic antiviral effects of the foam adjuvant agents, i.e., fatty alcohols and acids, provides a combined effect that should result in a better therapeutic response to treatment.

### Antiinflammatory or Antiallergic Agents

Yet, according to another embodiment according to the present invention the drug is an antiinflammatory or antiallergic agent. Antiinflammatory or antiallergic agent can be selected from the group of corticosteroids, non-steroidal antiinflammatory drugs (NSAIDs), anti-histamines, immunosuppressants and any combination thereof at a therapeutically effective concentration.

The following table provides a summary of currently available corticosteroid agent and their typical therapeutically effective concentration.

| Potency | Compound | Formulation |
|---|---|---|
| Very high | Clobetasol proprionate | Cream or ointment (0.05%) |
| | Halobetasol proprionate | Cream or ointment (0.05%) |
| High | Betamethasone diproprionate | Cream or ointment (0.05%) |
| | Betamethasone valerate | Ointment (0.1%) |
| | Fluocinolone actinide | Cream (0.02%) |
| | Halcinonide | Cream or ointment (0.1%) |
| Medium | Beamethasone valerate | Cream (0.1%) |
| | Fluocinolone acetonide | Cream or ointment (0.02%) |
| | Hydrocortisone valerate | Cream or ointment (0.2%) |
| | Triamcinolone acetonide | Cream, ointment, or lotion |
| | | (0.1% or 0.02%) |
| Low | Hydrocortisone | Cream, ointment, or lotion |
| | | (1% or 2.5%) |

The concentrations of corticosteroid drugs, as presented in the above table are provided herein only as example, and any therapeutically effective concentration of such corticosteroids can be incorporated in the composition of the present invention.

Since all corticosteroid drugs are typically hydrophobic, the carrier of the present invention, comprising a hydrophobic solvent, is most suitable as a vehicle to facilitate better topical distribution and an enhanced rate of penetration of any of the above listed drugs. Furthermore, the intrinsic antiviral, antibacterial and antiinflammatory effects of the foam adjuvant agents, i.e., fatty alcohols, provides a combined effect that should result in a better therapeutic response to treatment.

Psoriasis is a very common chronic skin disease, which may be the target of treatment using the composition of the present invention. It is marked by periodic flare-ups of sharply defined red patches covered by a silvery, flaky surface.

Corticosteroid ointments, greasy preparations containing little or no water, are commonly used for treating psoriasis. Their main disadvantage is in their sticky feeling, which remains so long after treatment is over. By contrast, the foam of the present invention, while comprising considerable concentration of an oil (hydrophobic solvent), spreads very easily throughout the afflicted area and absorbs into the skin without leaving any untoward sensation or look. Examples of other inflammatory disorders, which can be treated by the composition of the present invention, wherein the drug is a steroid are atopic dermatitis, seborrhea, seborrheic dermatitis of the face and trunk, seborrheic blepharitis, contact dermatitis, stasis dermatitis (gravitational eczema; varicose eczema), exfoliative dermatitis (erythroderma), lichen simplex chronicus, pityriasis rosea and pemphigus.

Topical antihistaminic preparations currently available include 1% and 2% diphenhydramine (Benadryl® and Caladryl®), 5% doxepin (Zonalon®) cream, phrilamine maleate, chlorpheniramine and tripelennamine, phenothiazines, promethazine hydrochloride (Phenergan®) and dimethindene maleate. These drugs, as well as additional antihistamins can also be incorporated in the composition of the present invention.

It is pointed out that polyunsaturated fatty acids, containing omega-3 and omega-6 fatty acids (e.g., linoleic and linolenic acid, gamma-linoleic acid (GLA), eicosapentaenoic acid (EPA) and docosahexaenoic acid (DHA) are beneficial in the treatment of psoriasis and other skin inflammation conditions.

A second class of anti-inflammatory agents, which is useful in the foam of the present invention, includes the nonsteroidal anti-inflammatory agents (NSAIDs). The variety of compounds encompassed by this group is well-known to those skilled in the art. Specific non-steroidal anti-inflammatory agents useful in the composition invention include, but are not limited to:
1) Oxicams, such as piroxicam, isoxicam, tenoxicam, sudoxicam;
2) Salicylates, such as salicylic acid, ethyl salicylate, methyl salycilate, aspirin, disalcid, benorylate, trilisate, safapryn, solprin, diflunisal, and fendosal;
3) Acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;
4) Fenamates, such as mefenamic, meclofenamic, flufenamic, niflumic, and tolfenamic acids;
5) Propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and
6) Pyrazoles, such as phenylbutazone, oxyphenbutazone, feprazone, azapropazone, and trimethazone.

Any further steroidal and nonsteroidal compounds, having the capacity to prevent, alleviate the symptoms of, treat or cure inflammation processes, are generally included, as possible anti-inflammatory agents, according to the present invention.

The pharmaceutical composition of the present invention may also comprise an antiinflammatory or antiallergic agent, wherein said agent reduces the occurrence of pro-inflammatory cytokines or inhibits the effect of pro-inflammatory cytokines.

Mixtures of such anti-inflammatory agents may also be employed, as well as the dermatologically acceptable salts, esters, amides, prodrugs and derivatives of these agents.

Topical application of a foam, comprising a safe and effective dose of an NSAID can be useful in the prevention and/or alleviation of the symptoms of rheumatoid arthritis, osteoarthritis and pain. Topical NSAIDs, incorporated in the foam of the present invention can be also used in the treatment of dermatological disorders, such as acne, rosacea, hair growth disorders, actinic keratosis and certain skin cancer conditions.

### Topical Anesthetics

The compositions of the present invention may contain a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof. Mixtures of such anesthetic agents may be synergistically beneficial.

Keratolytically Active Agents The term “keratolytically active agent” is used herein to mean a compound that loosens and removes the stratum corneum of the skin, or alters the structure of the keratin layers of skin.

Keratolytically active agents are used in the treatment of many dermatological disorders, which involve dry skin, hyperkeratiinization (such as prsoriasis), skin itching (such as xerosis), acne and rosacea.

Suitable keratolytically active agent include phenol and substituted phenolic compounds. Such compounds are known to dissolve and loosen the intracellular matrix of the hyperkeratinized tissue. As such, they are used in the treatment of dermatological disorders. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic. These compounds also exhibit antiseptic properties. Cresols also possess bactericidal and keratolytic properties.

Vitamin A and its derivatives, such as retinoic acid, isoretinoic acid, retinol and retinal are another preferred class of keratolytically active agents.

Another group of keratolytically active agents include alpha-hydroxy acids, such as lactic acid and glycolic acid and their respective salts and derivatives; and beta-hydroxy acids, such as Salicylic acid (o-hydroxybenzoic acid) and its salts and pharmaceutically acceptable derivatives, which typically possess antiinflammatory, as well as keratolytic, activity.

Yet, another class of preferred keratolytically active agents includes urea and its derivatives.

### Retinoids

Another preferred group of active agents comprise retinol, retinal, all trans retinoic acid and derivatives, isomers and analogs thereof, collectively termed “retinoids”. Etretinate, actiretin, isotretinoin, adapalene and tazarotene are further examples of said retinoid isomers and analogs. Compositions according to the present invention, which contain retinoids as the active drug, can be used for the treatment of acne, seborrhea, various dermatoses, inflammation of the skin, mucosal membranes, vagina and the rectum, psoriasis, actinic keratosis and skin cancers, by application onto the affected area.

### Insecticide and Insect Repellents Agents

Insects, such as mosquitoes, biting flies, mites, gnats, fleas, chiggers, punkies, sand flies, lice and ticks can be annoying and sometimes pose a serious risk to human and animal health. In certain areas of the United States, mosquitoes can transmit diseases like equine and St. Louis encephalitis. Biting flies can inflict a painful bite that can persist for days, swell, and become infected. Ticks can transmit serious diseases like Lyme disease and Rocky Mountain spotted fever.

There are several types of insect repellents to use when protecting people and animals from flying or biting insects, spiders, ticks and mites. By way of example, these may include DEET (N, N-diethyl-m-toluamide), dimethyl phthalate, piperonyl butoxide and permethrin. Insect repelling terpenoids, have been reported by Hwang, et al, J. Chem. Ecol., 11, 1297 (1-985); and Ruledge, J. Am. Mosquito Control Assoc. 4, 414 (1988).

A particularly preferred group of insect repellents includes the terpenoid compounds, described in U.S. Pat. No. 5,411,992 (incorporated by reference), including:
(1) Terpenoid-alcohol or terpene-ols are terpenoids that have at least one hydroxyl group. Examples of terpene-ols include: C10H16O compounds, perillyl alcohol, carveol, myrtenol, and cis-verbenol; C10H18O compounds, myrtanol, iso-pinocampheol, dihydrocarveol, isopulegol, terpineol, terpinen-4-ol, nerol, geraniol, and linalool, and C10H20O compounds, menthol, beta-citronellol, and dihydro-myrcenol;
(2) Terpenoid-esters are terpenoids, which have at least one ester group that is the product of the bonding of the hydroxyl group of a terpene-ol with an aliphatic carboxylic acid that can contain functional groups such as the hydroxyl or amine on the aliphatic chain. Examples of suitable aliphatic carboxylic acids include acetic acid, propionic acid, lactic acid, and various amino acids. Examples of terpenoid-esters include: carvyl acetate, carvyl propionate, and menthyl lactate; and
(3) Essential oils which contain terpenoids and perfumes which contain terpenoids. Non-limiting examples of essential oils which have high content of terpene-ols and esters include bergamot (62% terpenoids); sage (>50% terpenoids); styrax (>50% terpenoids); peppermint (>50% terpenoids); and pine Siberian (75% terpenoids %). Terpenes, aldehydes and ketones vary in their usefulness but as a general group have potential as insect-repellent.

The foam of the present invention is particularly suitable for the effective uniform spreading of an insect repellent agent onto large areas of the skin of humans and animals. The hydrophobic solvent present in the foam composition helps retain the insect repellent on the skin surface for an extended period of time.

Yet, in a further embodiment, the foam is suitable for delivery of insect-killing agents (insecticides) to an afflicted external surface area of humans and animals. Thus, the pharmaceutical or cosmetic composition may comprise an insecticide, known in the art of parasitology. By way of example, such insecticide can be selected from the group of permethrin, hexachlorobenzene, carbamate, naturally occurring pyrethroids, permethrin, allethrin, malathion, piperonyl butoxide and any combination thereof at a therapeutically effective concentration. Its application is very convenient and it spreads easily, even over hairy areas. The hydrophobic solvent present in the foam composition helps retain the insecticide on the treated area for an extended period of time. Furthermore, the presence of a hydrophobic solvent in the foam eases mechanical removal of lice and nits with a comb.

### Anticancer Agents

Generally speaking, anticancer agents can also be used according to the present invention as the drug of choice from skin malignant tumors, such as basal cell carcinoma, squamous cell carcinoma, melanoma and Kaposi's sarcoma, as well as the pre-cancerous condition actinic keratosis. In certain cases, topical cytotoxic and antiproliferative drugs are used to treat or prevent such cancers, including 5-fluorouracil (5-FU), as well as any other anti-cancer agents, can be also incorporated into the foam at therapeutically effective levels.

### Photodynamic Therapy Agents

The foam composition of the present invention is also useful to deliver photo-sensitizing agents, known in the art of photodynamic therapy. By way of example, such photosensitizers can be selected from the group comprising modified porphyrins, chlorins, bacteriochlorins, phthalocyanines, naphthalocyanines, pheophorbides, purpurins, m-THPC, mono-L-aspartyl chlorin e6, bacteriochlorins, phthalocyanines, benzoporphyrin derivatives, as well as photosensitiser precursors, such as aminolevulinic acid (ALA).

### Active Agents for Burns, Wounds, Cuts and Ulcers

The treatment of burns, wounds, cuts and ulcers, using the composition of the present invention is particularly advantageous. The foam can include both anti-infective agents (against bacteria, fungi and/or viruses), antiinflammatory agents (steroidal and/or NSAIDs) and pain relieving components. Upon application, the foam spreads easily, covering the surface of the affected area, and without causing pain.

### Skin Care Active Agents

The foam of the present invention is useful and advantageous for skin care and cosmetic care. The combination of oil and water, having moisture-retaining properties, in a spreadable foam form, can be used to substitute currently used cosmetic skin care creams, lotions, gels, etc. The cosmetic foam compositions of the present invention are suitable for the further application as “cosmeceutical” preparation (cosmetic products with therapeutic benefit), to treat “cosmetic” skin disorders, such as aging skin, wrinkles, hyperpigmentation (melasma, chloasma, freckles, etc.), scaly skin and other skin undesirable properties.

The CTFA Cosmetic Ingredient Handbook describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, astringents, etc. (e.g., clove oil, menthol, camphor, eucalyptus oil, eugenol, menthyl lactate, witch hazel distillate), anti-acne agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives thereof.

In any embodiment of the present invention, however, the active agents useful herein can be categorized by the benefit they provide or by their postulated mode of action. It is to be understood that the active agents useful herein can in some instances provide more than one benefit or operate via more than one mode of action. Therefore, classifications herein are made for the sake of convenience and are not intended to limit the active to that particular application or applications listed.

### Anti-Acne Agents

The compositions of the present invention may contain a safe and effective amount of one or more pharmaceutically or cosmetically acceptable anti-acne active agents. Examples of useful anti-acne actives include resorcinol, sulfur, salicylic acid and salicylates, alpha-hydroxy acids, nonsteroidal anti-inflammatory agents, benzoyl peroxide, retinoic acid, isoretinoic acid and other retinoid compounds, adapalene, tazarotene, azelaic acid and azelaic acid derivatives, antibiotic agents, such as erythromycin and clyndamycin, zinc salts and complexes, and combinations thereof, in a therapeutically effective concentration.

### Antiwrinkle and Antiatrophy Agents and Agents to Treat Dry and Scaly Skin (E.g., Xerosis and Ichthyosis)

The compositions of the present invention may further contain a safe and effective amount of one or more anti-wrinkle actives or anti-atrophy actives, which can be easily delivered by spreading a foam onto the skin. Exemplary anti-wrinkle/anti-atrophy active agents suitable for use in the compositions of the present invention include sulfur-containing D and L amino acids and their derivatives and salts, particularly the N-acetyl derivatives; thiols; hydroxy acids (e.g., alpha-hydroxy acids such as lactic acid and glycolic acid and their derivatives and salts; or beta-hydroxy acids such as salicylic acid and salicylic acid salts and derivatives), urea, hyaluronic acid, phytic acid, lipoic acid; lysophosphatidic acid, skin peel agents (e.g., phenol, resorcinol and the like), vitamin B3 compounds (e.g., niacinamide, nicotinic acid and nicotinic acid salts and esters, including non-vasodilating esters of nicotinic acid (such as tocopheryl nicotinate), nicotinyl amino acids, nicotinyl alcohol esters of carboxylic acids, nicotinic acid N-oxide and niacinamide N-oxide), vitamin B5 and retinoids (e.g., retinol, retinal, retinoic acid, retinyl acetate, retinyl palmitate, retinyl ascorbate). In the case of dry, scaly skin (xerosis) and ichthyosis such agents can alleviate the symptoms by temporary relief of itching associated with these conditions.

### Antioxidants and Radical Scavengers

A safe and effective amount of an anti-oxidant/radical scavenger may be added to the compositions of the subject invention, preferably from about 0.1% to about 10% (w/w), more preferably from about 1% to about 5% (w/w), of the composition.

Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox.sup.R), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used.

The foam of the present invention is suitable for delivering skin protecting and revitalizing anti-oxidants/radical scavengers. It is further pointed out that polyunsaturated fatty acids, containing omega-3 and omega-6 fatty acids (e.g., linoleic and linolenic acid, gamma-linoleic acid (GLA), eicosapentaenoic acid (EPA) and docosahexaenoic acid (DHA) are beneficial in the treatment of psoriasis and other skin inflammation conditions. Likewise, emollients and silicone oils exert moisture-retaining and skin protective effects on the skin. Thus in a preferred embodiment, a skin protective foam is provided, wherein the hydrophobic solvent comprises in full or in part, a solvent, selected from the group of emollients, silicone oil and oils, rich in unsaturated fatty acids, thus, affording a synergistic therapeutic effect of the anti-oxidants/radical scavenger agent and the vehicle components.

### Self-Tanning Agents

The foam of the present invention is particularly suitable for the uniform delivery of a tanning active agent onto large areas of the skin. It is preferable that the compositions contain, for example from about 0.1% to about 20%, from about 2% to about 7%, or from about 3% to about 6%, of the composition, of dihydroxyacetone, or any other compound, known in the art as an artificial tanning active agent.

### Skin-Lightening and Skin-Whitening Agents

The foam of the present invention is particularly suitable for the uniform delivery of a skin-lightening agent. When used, the compositions may contain, for example, from about 0.1% to about 10% or from about 0.2% to about 5% of the composition, of a skin-lightening agent. Suitable skin lightening or whitening agents include those known in the art, including hydroquinone, azelaic acid and other related dicarboxylic acids, and salts and derivatives thereof, retinoids, kojic acid, arbutin, nicotinic acid and its precursors, salts and derivatives, arbutin, ascorbic acid and salts and derivatives thereof (e.g., magnesium ascorbyl phosphate or sodium ascorbyl phosphate), and herbal extracts (e.g., licorice extract, mulberry extract, placental extract).

In one or more embodiments of the present invention, the foam composition comprises a combination of a skin whitening agent and a sunscreen agent.

In one or more embodiments of the present invention, the foam composition comprises a combination of a skin whitening agent and an inorganic sunscreen agent. When inorganic sunscreen agents, e.g. TiO2, are rubbed onto the skin, they leave a white coating, which provides an immediate (although transient) whitening effect, which is highly desirable by the consumer, who wishes to see instant change in his/her appearance. The whitening agent, in combination with the inorganic sunscreen agent in the foam carrier can be easily and uniformly distributed on the skin surface, thereby affording an even instant whitening effect, unlike creams that are difficult to spread evenly on skin areas.

### Sunscreens

Exposure to ultraviolet light can result in excessive scaling and texture changes of the stratum corneum. The foam of the present invention is advantageous for the delivery of sunscreen agents. Its application is very convenient and it spreads easily over large skin areas. The presence of a hydrophobic solvent in the foam ensures long lasting effect, even while bathing.

As used herein, “sunscreen active” or “sunscreen agent” includes both sunscreen agents and physical sunblocks. Suitable sunscreen actives may be organic or inorganic.

Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens may be present in the amount of, for example, from about 0.1% to about 20%, from about 0.5% to about 10%, or from about 1% to about 5%, of the composition.

A wide variety of conventional organic sunscreen actives are suitable for use herein. Specific suitable sunscreen actives include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphthol sulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′, 4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane.

A safe and effective amount of the organic sunscreen active is used, typically from about 1% to about 20%, more typically from about 2% to about 10% of the composition. Exact amounts will vary depending upon the sunscreen or sunscreens chosen and the desired Sun Protection Factor (SPF).

### Agents for Hair-Growth Disorders

Agents, which affect the pattern of hair growth, can be suitably incorporated in the foam of the present invention. Male pattern baldness (MPB), the commonest cause of balding, is induced by the activity of the male hormone dihydrotestosterone (DHT), which converted from the hormone testosterone by the enzymes 5 alpha reductase. Current treatments of MPB include minoxidil and agents, which inhibit 5 alpha reductase, such as finasteride, spironolactone, azelaic acid and azelaic acid derivatives and salts. Such agents, as well as other agents known in the art, can be incorporated in the foam of the present invention.

It is further pointed out that polyunsaturated fatty acids, i.e., such which include any of the essential fatty acids (EFA's): linoleic and linolenic acid, gamma-linoleic acid (GLA), eicosapentaenoic acid (EPA) and docosahexaenoic acid (DHA), are also known to contribute to hair growth. Thus in a preferred embodiment, a hair growth foam is provided, wherein the hydrophobic solvent comprises in full or in part, an oil, rich in such unsaturated fatty acids.

### Figure-forming Agents and Agents to Treat Cellulite/Slimming

Figure forming agents such as used in the treatment of cellulite and in slimming products, can be suitably incorporated in the foam of the present invention. A non-limiting exemplary list of active agents, known in the treatment of cellulite and in the induction of a slimming effect include herbal extracts, such as baldderwack extract, butcher's, broom, cayenne, dandelion, red clover, ginkgo biloba, horse chestnut, witch hazel and borage oil, omega 3 and omega 6 oils, caffeic acid and salts and derivatives thereof, xanthine agents, such as caffeine, theophiline and pentoxyphilline, and nicotinic acid and salts and derivatives thereof.

### Agents to Treat Sunburn, Heat Burn, Radiation Burn, Rash and Itchiness

Cosmetic and pharmaceutical ingredients which are known in the art of pharmacology and cosmetology to treat dermatitis, minor skin irritations, sunburn, heat burn, radiation burn, and inhibit inflammation can be beneficially incorporated in the foam of the present invention.

Examples of such active agents include chamomile extract (matricaria recutitia), cucumber distillate (cucumis sativus), lavender water (lavendula angustifolia), rose water (rosa damascena), witch hazel (hamamelis virginiana), allantoin, bisabolol, rosehip oil, calendula oil, azulaene, menthol and camphor.

### Use of the Foam as a Lubricating and Protective Foam

There are several potential uses of the foam, particularly the silicone-oil based foam, as a lubricating foam. Typical examples are shaving foam, moisture protection foam and antifriction foam. For such purposes, the foam can be used in its basic composition (without additional formulation aids and active ingredients), or with the addition of such additives.

Foam for Neutralization and/or Decontamination of Hazardous Chemicals and Treatment of Heat Burns

It has been reported that povidone iodine antiseptic, a popular iodine product, can ameliorate damage to guinea pig skin exposed to mustard gas and other chemical irritants and further reduces, and many times prevents, damage to human skin after accidental heat burns caused by hot water, oil or hot steam.

Other active compound, having decontamination abilities, comprise strong oxidants and free radical liberating compounds, such as hydrogen oxide, bleaching agents (e.g., sodium, calcium or magnesium hypochloride and the like) iodine, chlorohexidine and benzoyl peroxide.

The alcohol-free foam of the present invention, comprising one or more of the above decontaminating and neutralizing agents can be applied onto the contaminated skin to form a preventive layer, prior to contamination measure or as a decontamination/neutralization means, right after contamination has occurred.

### Penetration Enhancers

A penetration enhancer or permeation enhancer is an agent used to increase the permeability of the skin to a pharmacologically active agent to increase the rate at which the drug diffuses through the skin and enters the tissues and bloodstream. A chemical skin penetration enhancer increases skin permeability by reversibly altering the physiochemical nature of the stratum corneum to reduce its diffusional resistance. In a review of the technical and patent literature up to 1996, numerous chemical compounds were cited as skin penetration enhancers. Most of the compounds are generally recognized as safe (GRAS) ingredients that would often be considered inert by a formulator (Osborne D W, Henke J J, Pharmaceutical Technology, November 1997, pp 58-86.)

Examples of penetration enhancers, according to the present invention include: polyols, such as propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, and glycerol; sulfoxides, such as dimethylsulfoxide (DMSO), dimethylformanide, methyl dodecyl sulfoxide, dimethylacetamide; monooleate of ethoxylated glycerides (with 8 to 10 ethylene oxide units); Azone (1-dodecylazacycloheptan-2-one), 2-(n-nonyl)-1,3-dioxolane; esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various surfactants, such as sodium lauryl sulfate; various alkanoic acids such as caprylic acid; lactam compounds, such as azone; alkanols, such as oleyl alcohol; dialkylamino acetates, and admixtures thereof.

Lower alcohols, such as ethanol, propanol, isopropanol, butanol, iso-butanol, t-butanol and pentanol, are not considered appropriate penetration enhancers according to the present invention, due to their skin drying and irritation properties.

Yet, another preferred class of penetration enhancers in the cyclodextrines and related compounds. Cyclodextrins are structurally related cyclic oligomaltoses that form a new group of pharmaceutical excipients. These are torus-shaped molecules with a hydrophilic outer surface and a lipophilic central cavity. Cyclodextrins are capable of forming water-soluble inclusion complexes with a wide variety of lipophilic water-insoluble drugs by taking up a whole drug molecule, or some part of it, into the cavity. The cyclodextrin molecules are relatively large (molecular weight ranging from almost 1000 to over 1500), with a hydrated outer surface, and under normal conditions, cyclodextrin molecules will only permeate the skin barrier with considerable difficulty. It is generally believed that the cyclodextrin molecules act as true carriers by keeping lipophilic drug molecules in solution and deliver them to the skin surface where they partition from the cyclodextrin cavity into the skin.

### INCORPORATION BY REFERENCE

All U.S. and PCT patent application publications and U.S. patents mentioned herein are hereby incorporated by reference in their entirety as if each individual patent application publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A composition, comprising:
(a) 88-97 wt % of a heavy cream product; and
(b) 3-12 wt % of a liquefied or compressed gas propellant;
wherein the heavy cream product and the liquefied or compressed gas propellant are admixed.

2. The composition of claim 1, wherein the heavy cream product comprises an oil-in-water emulsion.

3. The composition of claim 2, wherein the oil-in-water emulsion comprises:
(i) a high emollient, high viscosity cream, lotion, or ointment; and
(ii) water.

4. The composition of claim 3, wherein the oil-in-water emulsion comprises from 20%-70% water.

5. The composition of claim 3, wherein the high emollient, high viscosity cream, lotion, or ointment comprises a natural oil, a synthetic oil, or a combination thereof.

6. The composition of claim 3, wherein the high emollient, high viscosity cream, lotion, or ointment comprises one or more fatty alcohols.

7. The composition of claim 3, wherein the high emollient, high viscosity cream, lotion, or ointment comprises polyalkylene glycol monoalkyl ether, phosphates thereof, alkylene oxide polyol adducts, or a combination thereof.

8. The composition of claim 3, wherein the oil-in-water emulsion the emollient is ethylhexyl palmitate, coco-caprylate/caprate, C₁₂-C₁₅-alkyl ethylhexanoate, isononyl isononanoate, or a combination thereof.

9. The composition of claim 3, wherein the oil-in-water emulsion further comprises a surfactant.

10. The composition of claim 9, wherein the surfactant is an ionic surfactant, a nonionic surfactant, or a combination thereof.

11. The composition of claim 9, wherein the surfactant is an anionic surfactant or a cationic surfactant.

12. The composition of claim 9, wherein the surfactant is cetostearyl alcohol, polysorbate 60, PEG-150 stearate or steareth-20, or a combination thereof.

13. The composition of claim 1, wherein the liquefied or compressed gas propellant is a flammable propellant.

14. The composition of claim 13, wherein the flammable propellant is a volatile hydrocarbon or a volatile ether.

15. The composition of claim 14, wherein the flammable propellant is a volatile hydrocarbon; and the volatile hydrocarbon is propane, n-butane, isobutane, isobutene, isopentane, or a combination thereof.

16. (canceled)

17. The composition of claim 14, wherein the flammable propellant is a volatile ether; and the volatile ether is dimethyl ether or methyl ethyl ether.

18. The composition of claim 1, wherein the liquefied or compressed gas propellant is a non-flammable propellant.

19. The composition of claim 1, wherein the liquefied or compressed gas propellant is a green propellant.

20. The composition of claim 19, wherein the green propellant is a hydrofluoroalkane or a hydrofluoroalkene.

21. The composition of claim 20, wherein the hydrofluoroalkane is 1,1,1,2,3,3,3-heptafluoropropane or 1,3,3,3-tetrafluoropropene.

22. The composition of claim 1, wherein the composition is self-foaming.

23. A packaged composition, comprising a dispenser; and the composition of claim 1; wherein the composition is contained within the dispenser; and the composition forms a non-flammable foam upon dispensing.

24-31. (canceled)

32. A method for dispensing a self-foaming composition from the packaged composition of claim 23, comprising releasing the composition from the dispenser, wherein the composition forms a non-flammable foam upon dispensing.

33. The composition of claim 6, wherein the fatty alcohol is cetostearyl alcohol, cetyl stearyl alcohol, cetyl alcohol, or myristyl alcohol.
